(19) United States
(12) Patent Application Publication
(10) Pub. No.: US 2025/0319049 A1
(43) Pub. Date: Oct. 16, 2025

(54) Additive for animal nutrition

(71) Applicant: ADISSEO FRANCE S.A.S., ANTONY (FR)

(72) Inventors: Antoine PETRELLI, BRIANÇON (FR); Virginie BELLIERE-BACA, MILLERY (FR); Patrick REY, LYON (FR); Maxime HUCHEDE, CHAPONNAY (FR); Virginie MICHEL, Montluçon (FR); Jessika CONSUEGRA BONILLA, Chaponost (FR)

(21) Appl. No.: 18/881,352

(22) PCT Filed: Jul. 4, 2023
(86) PCT No.: PCT/FR2023/051023

§ 371 Date: Jan. 6, 2025

(30) Foreign Application Priority Data
Jul. 5, 2022 (FR) FR22/06816

(51) Int. Cl. A61K 31/19

(52) U.S. Cl. CPC A61K 31/19

(57) ABSTRACT

The disclosure concerns a use of a compound for the preparation of an additive, a premix or a food or fodder, for animal feeding, said compound corresponding to the following formula:

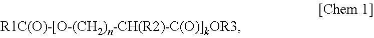

in which
R1 is selected from C₁₋₁₁ alkyl groups;
R2 is selected from C₁₋₂₀ alkyl groups; CₘH₂ₘ—OR4; CₘH₂ₘ—NHR4 where R4 is selected from H and C(O)—C₁₋₁₁ alkyl and m is equal to 0 or represents an integer from 1 to 11;
R3 is selected from H and C₁₋₂₀ alkyl groups;
n is 0 or an integer from 1 to 10; and
k is an integer from 1 to 10;
or a salt of said compound.

### CROSS-REFERENCE TO RELATED APPLICATIONS

This application is a National Stage application of PCT/FR2023/051023, filed Jul. 4, 2023, which claims the benefit of France Application No. FR22/06816 filed Jul. 5, 2022, both of which are incorporated by reference in their entirety herein.

### TECHNICAL FIELD

The present disclosure concerns the use in animal nutrition of compounds as sources of organic acids, as well as an additive for animal nutrition.

### BACKGROUND

The animal production depends on the use of appropriate and good quality feed. The additives are compounds intended to be incorporated into this feed in order to give it improved properties both from the point of view of the feed itself and with respect to the animals thus fed, or even the food products obtained from the latter. Among the additives for animal nutrition, there are compounds which have a favorable influence on the conservation, presentation, handling, and palatability of this feed. There are also nutritional compounds such as vitamins, amino acids, trace elements, as well as agents improving the digestibility, bioavailability of substances contained in the feed or in the additives.

European regulations [EC Regulation No. 1831/2003] prohibit the use of antibiotics, which has encouraged the development of natural substitutes with antibacterial properties. Organic acids, long used in food preservation, constitute an alternative whose use is becoming widespread in animal nutrition, and in particular butyric acid, lactic acid, propionic acid, acetic acid and formic acid. They are used as additives in animal nutrition, for the preservation of animal feed and its bacterial decontamination, but also in animal feed and drinking water, for their nutritional and health effects. Thus, butyric acid has both nutritional qualities and an antibacterial power that can act on the animal's gastrointestinal flora. Lactic acid is a bacteriostatic agent used as an additive. The disadvantage encountered by many active molecules is their fragility with respect to the environment of the digestive system of animals. As a result, they are released early in the upper intestine where they cross the intestinal barrier and quickly reach the blood, even before they can exert their beneficial effects on the intestinal flora in the lower intestine.

A protected form of butyric acid, tributyrin, or butyric acid triglyceride, is commercially available. It constitutes a supply of butyric acid in a form that is at least partially resistant to the aggressive conditions of the stomach.

The document CN 103082094 A provides a solution with a mixed triglyceride whose structure shown below combines two molecules of lactic acid and one molecule of butyric acid carried by an esterified glycerol molecule,

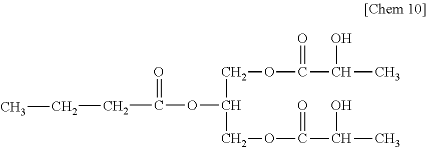 [SMILES: CCCC(=O)OC(COC(=O)C(C)O)COC(=O)C(C)O]

and which allows the progressive release of both butyric acid and lactic acid in the gastrointestinal tract of animals.

Just like tributyrin, it is observed that the release of lactic acid and/or butyric acid molecules occurs mainly in the upper part of the intestine and that only a tiny part of these molecules reaches the lower part of the intestine.

The administration of butyric acid and lactic acid, in the form of a triglyceride as mentioned above, provides the beginning of a solution.

But to date, there is a real need to have these molecules in particular, but also many other molecules, of the carboxylic acid type, of small sizes, having favorable effects on the microbiota, in a form that allows them to access the lower part of the intestine in order to exert their favorable effect on the intestinal microbiota.

### BRIEF SUMMARY

This is the problem that the present disclosure solves by providing a compound corresponding to the formula below:

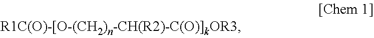

in which
R1 is selected from C₁₋₁₁ alkyl groups;
R2 is selected from C₁₋₂₀ alkyl groups; CₘH₂ₘ—OR4 and CₘH₂ₘ—NHR4 where R4 is selected from H and C(O)—C₁₋₁₁ alkyl and m is equal to 0 or represents an integer from 1 to 11;
R3 is selected from H and C₁₋₂₀ alkyl groups;
n is equal to 0 or represents an integer from 1 to 10; and
k represents an integer from 1 to 10;
or in a salt of said compound.

Thus the present disclosure concerns a use of a compound as defined above, for the preparation of an additive, a premix or a fodder feed, for animal nutrition, as well as an additive for animal feed consisting of such a compound.

According to a variant, the additive of the disclosure does not consist of butyryl lactylate, butyryl lactylate having the following formula:

[symbol]

It has indeed been demonstrated that the compound of formula [Chem 1], on the one hand, manages to reach the animal's intestine by having resisted the acidic conditions residing in the entire upstream part of the gastrointestinal tract, and on the other hand, allows the carried molecules to exert their benefits at the intestinal level.

According to the disclosure, the applications of a compound of formula [Chem 1] are not therapeutic, but nutritional, they target healthy animals. As an illustration with butyryl lactylate, once it reaches the intestine, the compound is hydrolyzed and releases butyrate which exerts numerous benefits for the intestine. It acts as a source of energy for intestinal cells, thus promoting their regeneration and proper functioning. In addition, butyrate exerts anti-inflammatory effects by reducing the production of pro-inflammatory cytokines, which contributes to the balance of the intestinal immune system. It also strengthens the intestinal barrier by promoting the production of protective mucus and improving the tight junction function between epithelial cells. Thus, butyrate plays a key role in promoting intestinal health and could have positive implications for general well-being.

### DETAILED DESCRIPTION

Before discussing the various subjects of the disclosure in more detail, certain terms used in the present text are defined.

A salt of the compound of formula [Chem 1] of the disclosure may be a salt of a carboxylic function of said compound to obtain a carboxylate function, a salt of a hydroxyl function, a salt of an amine function. It may be a combination of these possibilities.

By alkyl, is meant a CₓH₂ₓ₊₁ radical, aliphatic, which may include one or more non-aromatic rings; it may be linear or branched; by branched alkyl radical is meant a linear alkyl radical in which one or more carbon atoms are substituted by an alkyl group as defined above and itself, linear or branched.

In the definition of R2 in the formula [Chem 1] above, it should be understood that when it represents a CₘH₂ₘ—OR4 or CₘH₂ₘ—NHR4 group, the bond to the carbon bearing R2 is established at the Cₘ carbon of the CₘH₂ₘ—OR4 or CₘH₂ₘ—NHR4 group.

A compound of the disclosure corresponds to the formula [Chem 1] as defined above and in which one or more asymmetric carbon atoms may be present; it is understood according to the disclosure that the term «compound» covers all the enantiomers of said compound, alone or in a mixture.

A premix for animal nutrition, is a mixture of additives or a mixture of one or more additives with raw materials for animal feed or water used as a carrier, which are not intended for direct feeding of animals.

Thus, the disclosure concerns a use of a compound, for the preparation of an additive, a premix or a fodder feed, for animal nutrition, and an additive for animal nutrition consisting of a compound, said compound corresponding to the formula [Chem 1] above, or one of its salts.

According to a variant of the disclosure, this use is for feeding livestock. According to an implementation of this use, said compound, or its salt is intended for feeding monogastric livestock.

When the compound of formula [Chem 1] is in the form of a salt, it is advantageously a salt selected from alkali metal salts, alkaline earth metal salts, transition metal salts, ammonium salts and salts of ammonium derivatives. Advantageously, salts of Li, Na, K, Ca, Zn, Cu, Ni, Fe, Mn, Mg and Ag are selected.

It was unexpectedly discovered that the structure of the compound of formula [Chem 1] both has stability with respect to the deleterious environment of the animal's digestive system and allows the release of active molecules in the lower part of the intestine where they will exert their favorable effect on the intestinal microbiota.

Another advantage of a compound of formula [Chem 1] of the disclosure is to combine several molecules, identical or different, of interest in animal nutrition. For example, it may carry molecules of butyric acid, valeric acid and lactic acid, which are molecules well known for their benefits on intestinal microorganisms.

Thus, according to advantageous implementations of the disclosure which may be considered alone or in combination, the latter relates to the aforementioned use and to aforementioned additives concerning respectively the following compounds:
a compound having the formula [Chem 1] in which R1 is selected from methyl, ethyl, propyl, butyl, pentyl and hexyl groups;
a compound having the formula [Chem 1] in which R2 is selected from methyl, ethyl, propyl, butyl, pentyl and hexyl groups; OH; and O—C(O)-methyl, O—C(O)-ethyl, O—C(O)—propyl, O—C(O)-butyl, O—C(O)-pentyl and O—C(O)-hexyl groups;
a compound corresponding to the formula [Chem 1] in which n ranges from 0 to 2 and k ranges from 1 to 5.
butyryl lactylate corresponding to the following formula:

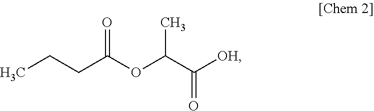

according to a variant, butyryl lactylate only concerns the use of the disclosure,
a compound corresponding to the following formula

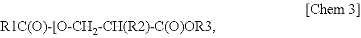

in which R2 is selected from CₘH₂ₘ—OR4 where m ranges from 0 to 2 and R4 is selected from H and C(O)—C₁₋₆ alkyl;
a compound corresponding to the following formula:

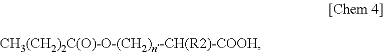

in which R2 is selected from OH; CH₂CH₂OH; and OC(O) CH₂CH₂CH₃; and n′ is equal to 0 or 1;
a compound corresponding to any one of the following formulae:

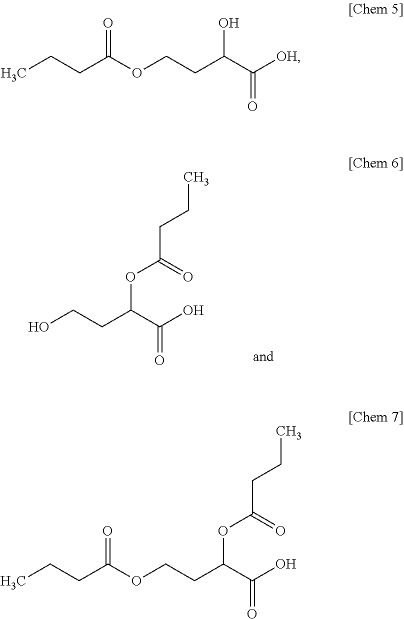 [SMILES: CCCC(=O)OCCC(O)C(=O)O ; CCCC(=O)OC(CCO)C(=O)O ; CCCC(=O)OCCC(OC(=O)CCC)C(=O)O]

The disclosure also concerns an aforementioned use and an aforementioned additive according to which the compound consists of any salt of a compound as defined below, or is in the form of any mixture of said compounds, said salts or said compounds and said salts.

The disclosure is of course not limited to these compounds, but they make it possible to support that the structure [Chem 1] is suitable for the optimized release, for example, of butyric acid on the one hand, and of a hydroxylated carboxylic acid having nutritional properties on the other hand, such as lactic acid, 2,4-dihydroxybutyric acid.

The disclosure also concerns any presentation and any use in animal nutrition of such an additive, as indicated below.

Thus, an object of the disclosure is a premix for animal feed including a mixture of additives, said premix including at least one additive of the disclosure as described above, and optionally a plant or mineral raw material.

Another object of the disclosure is a feed or fodder for animal nutrition which includes at least one additive of the disclosure or a premix of the disclosure as described above.

The disclosure further relates to the use in animal nutrition of a compound corresponding to the following formula:

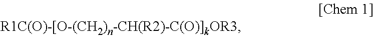

wherein
R1 is selected from C₁₋₁₁ alkyl groups;
R2 is selected from C₁₋₂₀ alkyl groups; CₘH₂ₘ—OR4; CₘH₂ₘ—NHR4; where R4 is selected from H and C(O)—C₁₋₁₁ alkyl and m is 0 or an integer from 1 to 11;
R3 is selected from H and C₁₋₂₀ alkyl groups;
n is 0 or an integer from 1 to 10; and
k is an integer from 1 to 10;
or a salt thereof.

In an advantageous use according to the disclosure, the compound is selected from the following compounds:
the compound corresponding to the following formula

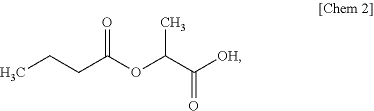

a compound corresponding to the following formula

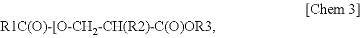

in which
R2 is selected from CₘH₂ₘ—OR4 where m ranges from 0 to 2 and R4 is selected from H and C(O)—C₁₋₆ alkyl;
a compound corresponding to the following formula

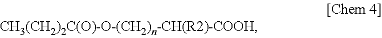

in which R2 is selected from OH; CH₂CH₂OH; and OC(O) CH₂CH₂CH₃ and n′ is equal to 0 or 1,
and the salts of said compound.

According to a variant of the disclosure, this use is aimed at feeding livestock. According to an implementation of this use, said compound, or its salt, is intended for feeding monogastric livestock.

In animal nutrition, said compound of the disclosure, or one of its salts, also has the advantage of being effective in any state whatsoever.

Thus, a compound of the disclosure such as butyryl lactylate can be incorporated into the drinking water of livestock, without risk of hydrolysis into lactic acid and free butyric acid in the water. Indeed, butyryl lactylate is soluble in water, whereas tributyrin is not.

A compound of the disclosure, such as butyryl lactylate, liquid, can be distributed in the form of a dry product, after dispersion on a support, in particular an inorganic support, such as a silica or calcium carbonate support. According to an embodiment, a compound of the disclosure such as butyryl lactylate can be dispersed or adsorbed on silica particles in a weight ratio in the range of 50 to 80% of butyryl lactylate and in the range of 20 to 50% of silica particles.

In another variant, a compound of the disclosure can also be administered in another solid form. For example, a butyryl lactylate salt, such as the calcium salt, is in the solid state. It can thus be distributed in the form of a dry product with a high butyric acid content, the butyric acid content in the butyryl lactylate salt being able to be comprised between approximately 1% by weight and approximately 60% by weight.

Moreover, butyric acid has a very unpleasant odor, which poses handling problems, whereas a compound of the disclosure is odorless. As demonstrated in an example below, it has been observed that butyryl lactylate has a palatability that contributes to the nutritional performance of the provided feed.

Thus, the disclosure concerns a use of a compound of [Chem 1], such as butyryl lactylate for the preparation of an additive, a premix or a fodder feed, for animal nutrition, for its palatability, that is to say its ability to attract the animal to consume it.

Another subject of the disclosure is a method for manufacturing a compound of the disclosure. This method uses reactive distillation. Thus, a mixture of the carboxylic acid molecules of interest is obtained and a reactive distillation is carried out to obtain a compound of formula [Chem 1]) of the disclosure.

Thus, the disclosure further concerns a method for manufacturing a compound of the disclosure or a salt of this compound, as defined above, including the following steps:
at least two molecules of carboxylic acids corresponding respectively to the following formulas are mixed

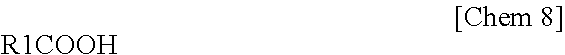

where R1 is selected from C₁₋₁₁ alkyl groups;

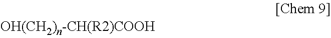

where n is equal to 0 or represents an integer from 1 to 10, and R2 is selected from C₁₋₂₀ alkyl groups; CₘH₂ₘ—OR4; CₘH₂ₘ—NHR4 where R4 is selected from H and C(O)—C₁₋₁₁ alkyl and m is equal to 0 or represents an integer from 1 to 11;
in the presence of a catalyst,
a reactive distillation is applied to the mixture thus obtained, and
a compound of formula [Chem 1] or a salt of this compound, corresponding to any one of the preceding definitions, is obtained.

In a typical reaction, molecules of carboxylic acids of interest are mixed with a suitable catalyst and the mixture is heated to a temperature which can vary from 25° C. to 150° C. The used catalyst is homogeneous or heterogeneous; when it is heterogeneous, it can be in a fixed bed (the reaction medium passes through the catalyst) or in a slurry (the catalyst is mixed with the reaction medium). A condenser or a distillation column can be attached to the reaction vessel so that water, any by-products and/or unreacted reagents can be removed from the vessel during the reaction. In addition, a vacuum can be applied to the reaction mixture to expel the water, said by-product(s) and/or unreacted reagent, more quickly and thus bring the reaction to completion more quickly. A third compound facilitating the removal of water from the reaction mixture can be added, such as toluene.

The reaction can be monitored, for example by TLC and/or GC and/or HPLC. If the reaction is complete, the reaction can be stopped by cooling the reaction mixture. If necessary, the catalyst is neutralized and filtered after the reaction and before or after purification. The compound thus obtained can further be purified by distillation or not.

As previously indicated, for animal feed, the compound can be used as such, dispersed on a support or in the form of a corresponding salt. The obtained salt can be optionally purified by washing, recrystallization, extraction with a solution to obtain a product having a higher purity.

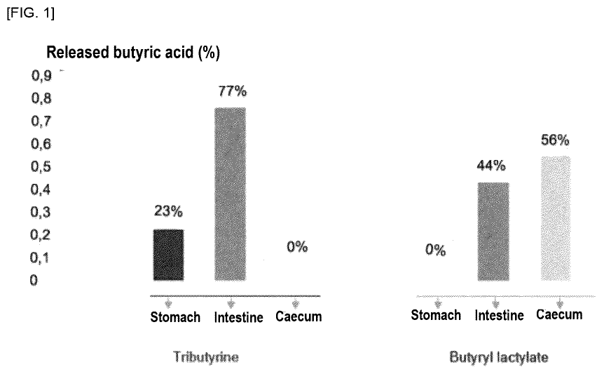

The present disclosure is illustrated in the following examples relating to the synthesis of compounds of the disclosure and their effects as an additive in animal nutrition and in support of [FIG. 1] which presents the content of butyric acid released (in % by weight relative to the weight of the tributyrin or butyryl lactylate administered), in the stomach and intestine defining the upper part of the gastrointestinal system, and the caecum defining the lower part of the gastrointestinal system.

### EXAMPLES

### Example 1: Preparation of Butyryl Lactylate from Lactic Acid and Butyric Acid in the Presence of Sulfuric Acid as Catalyst

1068.7 g (12 mol) of butyric acid (99%) are added to 151.9 g (1.52 mol) of a 90% (w/w) aqueous solution of lactic acid (90% w/w). 8 g (0.0775 mol) of sulfuric acid (95% w/w) are then added to the mixture. The mixture is brought to a temperature of 110° C. and the butyric acid/water azeotrope is distilled continuously. Vacuum is applied if necessary. After 5 h, the reaction mixture is cooled and 20.50 g (0.1538 mol) of an aqueous solution of sodium hydroxide (30% w/w) is added. The resulting precipitate is filtered. Excess water and butyric acid are removed by evaporation and 222.8 g of butyryl lactylate are obtained.

### Example 2: Preparation of Butyryl Lactylate Salt by Wet Process

946.6 g (0.63 mole) of a 5% w/w aqueous solution of calcium hydroxide are added to 208.9 g (1.26 mole) of a butyryl lactylate mixture as prepared in Example 1. The solid butyryl lactylate salt is obtained from the solution obtained by evaporation or spray-drying.

### Example 3: Preparation of Butyryl Lactylate Salt by Dry Process

63.06 g (0.63 mole) of solid calcium carbonate are mixed with 208.9 g (1.26 mole) of a butyryl lactylate mixture as prepared in Example 1. A neutralization reaction is carried out in an extruder to obtain the solid calcium salt of butyryl lactylate.

### Example 4: Preparation of Butyryl Lactylate Salt Conducted on Silica

208.9 g (1.26 mole) of butyryl lactylate as prepared in Example 1 are dispersed on 112.5 g of a suitable silica. The resulting preparation is dried to yield the solid butyryl lactylate salt.

### Example 5: Preparation of Butyryl Lactylate from Lactic Acid and Butyric Acid in the Presence of a Heterogeneous Catalyst

20.5 g (0.2303 mol) of butyric acid (99%) are added to 5.2 g (0.0556 mol) of lactic acid (98% w/w). 2 g of sulfonic acid resin are added to the mixture. The mixture is brought to a temperature of 110° C. and the butyric acid/water azeotrope is distilled continuously. Vacuum is applied if necessary. After 20 h, the reaction mixture is cooled and the resin is filtered. The excess butyric acid is removed by evaporation and 7.69 g of liquid butyryl lactylate are obtained.

### Example 6: Preparation of Butyryl Lactylate from Lactic Acid and Methyl Butyrate by Transesterification

22.45 g (0.22 mol) of methyl butyrate are added to 5 g (0.0544 mol) of L-lactic acid. 0.281 g (0.0027 mol) of sulfuric acid (95% w/w) are added to the mixture. The mixture is brought to a temperature of 90° C. Vacuum is applied if necessary. After 6 h, the reaction mixture is cooled, the excess methyl butyrate is removed by distillation and the butyryl lactylate is obtained.

### Example 7: Preparation of Butyryl Lactylate from Methyl Lactate and Butyric Acid by Transesterification

2.02 g (0.0192 mol) of methyl lactate are added to 6.79 g (0.0763 mol) of butyric acid. 0.11 g (0.0011 mol) of sulfuric acid (95% w/w) are added to the mixture. The mixture is brought to a temperature of 110° C. Vacuum is applied if necessary. After 6 h, the reaction mixture is cooled, the excess butyric acid is removed by distillation and the butyryl lactylate is obtained.

### Example 8: Preparation of Butyryl Lactylate from Lactic Acid and the Sodium Salt of Butyric Acid

3.01 g (0.0268 mol) of sodium butyrate are added to 0.449 g (0.0049 mol) of D,L-lactic acid. 1.74 g (0.0169 mol) of sulfuric acid (95% w/w) are added to the mixture. The mixture is brought to a temperature of 110° C. and the butyric acid/water azeotrope is distilled continuously. Vacuum is applied if necessary. After 5 h, the reaction mixture is cooled and the resulting precipitate is filtered. The excess water and butyric acid are removed by evaporation and 0.509 g of liquid butyryl lactylate are obtained.

### Example 9: Effects of Butyryl Lactylate-Comparison with Tributyrin

The experimental tests were conducted in in vitro models described below.

### 1) Upper Digestive Tract Model:

The method developed by Menezez and Blackburn was used to simulate the chicken digestive tract [Menezes-Blackburn D, Gabler S, Greiner R. «Performance of Seven Commercial Phytases in an in Vitro Simulation of Poultry Digestive Tract.»J Agric Food Chem. 2015 Jul. 15; 63 (27): 6142-9]. It consists of simulating the 3 compartments of the upper part of the digestive tract: the crop (30 min at pH 5, 40° C. with stirring), the proventriculus/gizzard (45 min at pH 3, 40° C. with stirring with the addition of pepsin), the intestine (60 min at pH 6.5, 40° C. with stirring with the addition of pancreatin and bile extract). The rate of protection and release of butyrate for each molecule (50 mM butyric acid equivalent of the butyryl lactylate of the disclosure or tributyrin of the prior art) was evaluated in each compartment sequentially (quantification of the release of butyric acid and residual butyric ester).

### 2) In Vitro Model: Lower Digestive Tract (Caecal Fermentation):

Twelve twenty-one-day-old broiler chickens (Ross PM33), housed in cages, fed and watered ad libitum, were euthanized and their caecal contents were collected and pooled. All experiments were conducted in accordance with the European Union animal protection guidelines and the legislation governing the ethical treatment of animals, and the researchers were certified by the French government to conduct animal experiments. The facilities of the Centre of Expertise and Research in Nutrition comply with Convention No. C 03 159 4 of 6 Nov. 2008, relating to experiments on living vertebrate animals (European Regulation 24/11/86 86/609 EEC; Ministerial Order of 19 Apr. 1988).

The cecal contents were mixed (5% w/v) with an anaerobic buffer prepared according to Theodorou et al. [Theodorou, Michael K., et al. “A simple gas production method using a pressure transducer to determine the fermentation kinetics of ruminant feeds.” Animal feed science and technology 48.3-4 (1994): 185-197], then the microbial suspension transferred into Hungate tubes containing 50 mM of butyric acid equivalent for each candidate (butyryl lactylate of the disclosure or tributyrin of the prior art) and incubated for 24 hours with constant stirring at 40° C.

To ensure that none of the treatments had a detrimental effect on microbial activity and viability, gas production was measured kinetically throughout the fermentation using a pressure transducer following the methodology of Theodorou et al.

At the end of the fermentation, the butyric acid and residual butyric ester were quantified.

### Example 10: Test Results

The results are presented on the FIGURE.

It is observed that butyryl lactylate does not release butyric acid in the stomach and an improved intestinal release profile with a balanced release in the upper and lower intestine unlike tributyrin which shows a significant gastric loss and a complete release at the beginning of the small intestine.

These results demonstrate the efficacy of a compound of the disclosure compared to tributyrin.

### Example 11: Evaluation of the Palatability of Butyryl Lactylate

This evaluation was carried out by in vivo tests conducted in broilers and in piglets at the post-weaning stage.

### 1) Tests in Broilers

The chickens were placed in normal breeding conditions.

They were divided into several groups, and each group received for five days a feed containing a source of butyrate, incorporated directly into a standard feed (STD). Several sources of butyrate were compared: the butyryl lactylate of the disclosure and two protected forms of butyrate, commercially available, a fat-coated butyrate and tributyrin.

The growth performances and feed consumption were measured at the beginning and end of the study.

The results are presented in Table 1 below.

TABLE 1
Effect of butyric acid sources compared to
a standard diet (%) in broiler chickens

| | BWG | FI | FCR |
|---|---|---|---|
| | 17-21 days | 17-21 days | 17-21 days |
| STD + fat coated butyrate | −5% | −15% | −10% |
| STD + tributyrine butyrate | −9% | −3% | +6.6% |
| STD + butyryl lactylate | +10% | +2.8% | −6.7% |

BWG: body weight gain
FI: feed intake
FCR: feed conversion ratio

Table 1 shows that the group of chickens fed existing protected butyrate sources (fat-coated and tributyrin) had a lower weight gain than chickens fed standard feed, −5% and −9% respectively (BWG). This lower growth is linked to a decrease in consumption of −15% and −3% (FI).

Although in protected form, it was noted that the feed containing fat-coated butyrate had a strong and unpleasant rancid odor, this bad odor could explain the decrease in consumption.

Conversely, the animals fed the feed with butyryl lactylate consumed more feed, +2.8% FI compared to the standard diet, thus increasing their growth (BWG) by +10%. The consumption of the feed with butyryl lactylate is higher than that of the standard feed, which allows us to conclude that butyryl lactylate encourages the animals to consume more.

### 2) Tests in Post-Weaning Piglets

The animals were placed in normal breeding conditions.

They were divided into four distinct groups, and each group received for about ten days (from 36 to 49 days of age) a standard feed (STD) containing a source of butyrate. Several sources of butyrate were compared: the butyryl lactylate of the disclosure and two protected forms of butyrate, commercially available, a coated butyrate (fat-coated) and tributyrin.

The growth performances and feed consumption were measured at the beginning and end of the study.

The results are presented in Table 2 below.

TABLE 2
Effect of butyric acid sources compared
to a standard control (%) in piglets

| | BWG | FI | FCR |
|---|---|---|---|
| | 36-49 days old | 36-49 days | 36-49 days |
| STD + fat coated butyrate | −31% | −13% | +26% |
| STD + tributyrine butyrate | −17% | −14% | +3.3% |
| STD + Butyryl lactylate | +21% | +16% | −4.8% |

BWG: body weight gain
FI: feed intake
FCR: feed conversion ratio

Table 2 shows that both sources of protected butyrate (fat coated and tributyrin) have a detrimental effect on animal performance. Compared to the standard group, animal growth (BWG) is degraded by −31% with fat-coated butyrate and by −17% with tributyrin. Feed efficiency (FCR) is also degraded, by +26% and +3.3% respectively. This drop in performance is linked to the significant drop in consumption, by −13% and −14% respectively.

It was noted that the feed containing fat-coated butyrate and the feed containing tributyrin have a strong odor, which leads to lower consumption. Indeed, by giving off an unpleasant and unpalatable odor, the animals were forced to spend less time at the feeder.

On the other hand, butyryl lactylate has a beneficial effect. The piglets that received the feed containing it consumed more feed than piglets receiving a standard commercial feed: +16% FI. Their growth (BWG) was favored by this increase in consumption, it is +21% higher. Feed efficiency was not impacted, it was even improved by −4% (FCR) with the feed containing butyryl lactylate. It therefore encouraged the animals to consume. This is a demonstration that butyryl lactylate has an appetizing aspect. In addition, it had an effect on digestive health, because the increase in consumption resulted in an improvement in BWG and FCR, this excess consumption was therefore valued by the animal.

This example demonstrates the appetizing effect of butyryl lactylate compared to other sources of butyrate, promoting the growth of animals without degrading feed efficiency.

1. A composition being an additive, a premix or a feed or fodder, for animal nutrition, comprising a compound, wherein said compound corresponds to the following formula: 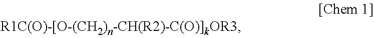
in which
R1 is selected from C₁₋₁₁ alkyl groups;
R2 is selected from C₁₋₂₀ alkyl groups; CₘH₂ₘ—OR4; CₘH₂ₘ—NHR4 where R4 is selected from H and C(O)—C₁₋₁₁ alkyl and m is equal to 0 or represents an integer from 1 to 11;
R3 is selected from H and C₁₋₂₀ alkyl groups;
n is equal to 0 or represents an integer from 1 to 10; and
k represents an integer from 1 to 10;
or in a salt of said compound.

2. The composition of claim 1, wherein the composition is an animal nutrition of monogastric livestock.

3. An animal feed additive consisting of a compound having the following formula: 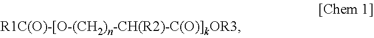
wherein
R1 is selected from C₁₋₁₁ alkyl groups;
R2 is selected from C₁₋₂₀ alkyl groups; CₘH₂ₘ—OR4; CₘH₂ₘ—NHR4 where R4 is selected from H and C(O)—C₁₋₁₁ alkyl and m is 0 or represents an integer from 1 to 11;
R3 is selected from H and C₁₋₂₀ alkyl groups;
n is 0 or represents an integer from 1 to 10; and
k represents an integer from 1 to 10;
or a salt of said compound,
said compound being different from the compound having the following formula

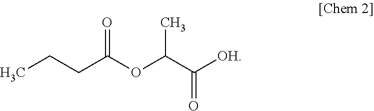

4. The composition of claim 1, wherein the compound corresponds to the formula [Chem 1] in which R1 is selected from methyl, ethyl, propyl, butyl, pentyl, hexyl groups, or a salt of said compound.

5. The composition of claim 1, wherein the compound corresponds to the formula [Chem 1] in which R2 is selected from methyl, ethyl, propyl, butyl, pentyl and hexyl groups; OH; and the O—C(O)-methyl, O—C(O)-ethyl, O—C(O)-propyl, O—C(O)— butyl, O—C(O)-pentyl and O—C(O)-hexyl groups, or a salt of said compound.

6. The composition of claim 1, wherein n ranges from 0 to 2 and k ranges from 1 to 5.

7. The composition of claim 1, wherein the compound corresponds to the following formula: 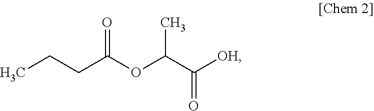
or a salt of said compound.

8. The composition of claim 1 wherein the compound corresponds to the following formula: 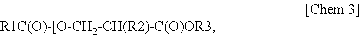
in which
R2 is selected from CₘH₂ₘ—OR4 where m ranges from 0 to 2 and R4 is selected from H and C(O)—C₁₋₆ alkyl,
or a salt of said compound.

9. The composition of claim 1, wherein the compound corresponds to the following formula: 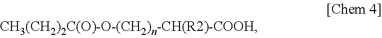
in which R2 is selected from OH; CH₂CH₂OH; and OC(O) CH₂CH₂CH₃ and n′ is equal to 0 or 1, or a salt of said compound.

10. The composition of claim 1, wherein the salt of formula [Chem 1] is selected from alkali metal salts, alkaline earth metal salts, transition metal salts, ammonium salts and salts of ammonium derivatives.

11. The composition of claim 10, wherein the salt of said compound is selected from the salts of Li, Na, K, Ca, Zn, Cu, Ni, Fe, Mn, Mg and Ag.

12. (canceled)

13. The composition of of claim 1, further comprising a vegetable or mineral raw material.

14. (canceled)

15. The additive of claim 3, wherein the compound corresponds to the formula [Chem 1] in which R1 is selected from methyl, ethyl, propyl, butyl, pentyl, hexyl groups, or a salt of said compound.

16. The additive of claim 3, wherein the compound corresponds to the formula [Chem 1] in which R2 is selected from methyl, ethyl, propyl, butyl, pentyl and hexyl groups; OH; and the O—C(O)-methyl, O—C(O)-ethyl, O—C(O)-propyl, O—C(O)-butyl, O—C(O)— pentyl and O—C(O)-hexyl groups, or a salt of said compound.

17. The additive of claim 3, wherein n ranges from 0 to 2 and k ranges from 1 to 5.

18. The additive of claim 3, wherein the compound corresponds to the following formula: 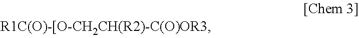
in which R2 is selected from CₘH₂ₘ—OR4 where m ranges from 0 to 2 and R4 is selected from H and C(O)—C₁₋₆ alkyl,
or a salt of said compound.

19. The additive of claim 3, wherein the compound corresponds to the following formula: 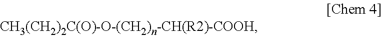
in which R2 is selected from OH; CH₂CH₂OH; and OC(O) CH₂CH₂CH₃ and n′ is equal to 0 or 1, or a salt of said compound.

20. The additive of claim 3, wherein the salt of formula [Chem 1] is selected from alkali metal salts, alkaline earth metal salts, transition metal salts, ammonium salts and salts of ammonium derivatives.

21. The additive of claim 20, wherein the salt of said compound is selected from the salts of Li, Na, K, Ca, Zn, Cu, Ni, Fe, Mn, Mg and Ag.

22. A method for improving the palatability of an additive, a premix or a fodder feed, for animal nutrition, comprising the addition to the additive, premix or fodder feed, of a compound corresponding to the following formula: 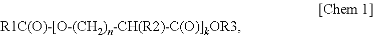
in which
R1 is selected from C₁₋₁₁ alkyl groups;
R2 is selected from C₁₋₂₀ alkyl groups; CₘH₂ₘ—OR4; CₘH₂ₘ—NHR4 where R4 is selected from H and C(O)—C₁₋₁₁ alkyl and m is equal to 0 or represents an integer from 1 to 11;
R3 is selected from H and C₁₋₂₀ alkyl groups;
n is equal to 0 or represents an integer from 1 to 10; and
k represents an integer from 1 to 10;
or in a salt of said compound.

23. The method of claim 22, wherein the compound corresponds to the formula [Chem 1] in which RI is selected from methyl, ethyl, propyl, butyl, pentyl, hexyl groups, or a salt of said compound.

24. The method of claim 22, wherein the compound corresponds to the formula [Chem 1] in which R2 is selected from methyl, ethyl, propyl, butyl, pentyl and hexyl groups; OH; and the O—C(O)-methyl, O—C(O)-ethyl, O—C(O)-propyl, O—C(O)-butyl, O—C(O)— pentyl and O—C(O)-hexyl groups, or a salt of said compound.

25. The method of claim 22, wherein in formula [Chem 1] n ranges from 0 to 2 and k ranges from 1 to 5.

26. The method of claim 22, wherein the compound corresponds to the following formula: 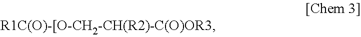
in which
R2 is selected from CₘH₂ₘ—OR4 where m ranges from 0 to 2 and R4 is selected from H and C(O)—C₁₋₆ alkyl,
or a salt of said compound.

27. The method of claim 22, wherein the compound corresponds to the following formula: 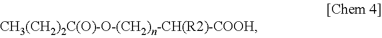
in which R2 is selected from OH; CH₂CH₂OH; and OC(O) CH₂CH₂CH₃ and n′ is equal to 0 or 1, or a salt of said compound.

28. The method of claim 22, wherein the salt of formula [Chem 1] is selected from alkali metal salts, alkaline earth metal salts, transition metal salts, ammonium salts and salts of ammonium derivatives.

29. The method of claim 22, wherein the salt of said compound is selected from
the salts of Li, Na, K, Ca, Zn, Cu, Ni, Fe, Mn, Mg and Ag.
